(19) United States
(12) Patent Application Publication
(10) Pub. No.: US 2024/0316437 A1
(43) Pub. Date: Sep. 26, 2024

(54) INTELLIGENT MOUTHGUARD

(71) Applicant: Trafton D. Rodgers, Lubbock, TX (US)

(72) Inventors: Trafton D. RODGERS, Lubbock, TX (US); Joseph S. WILLIAMSEN, Woods Cross, UT (US)

(73) Assignee: Trafton D. Rodgers, Lubbock, TX (US)

(21) Appl. No.: 18/275,128

(22) PCT Filed: Jan. 27, 2022
(86) PCT No.: PCT/US2022/014059

§ 371 Date: Jul. 31, 2023

Related U.S. Application Data
(60) Provisional application No. 63/143,683, filed on Jan. 29, 2021.

(51) Int. Cl. A63B 71/08; A61F 5/56

(52) U.S. Cl. CPC A63B 71/085; A61F 2005/563; A61F 5/566; A63B 2230/06; A63B 2230/40; A63B 2230/50

(57) ABSTRACT

A mouthguard has a body having a channel to receive a user's set of upper or lower teeth. The mouthguard has a processor, a power source, and a display embedded in the interior of the body.

### BACKGROUND

Athletes, particularly those participating in contact sports often wear a protective device for the mouth that covers the teeth and gums to prevent and reduce injury to the teeth, arches, lips and gums known as a mouthguard. The device may also be described as a mouth protector, mouth piece, gumshield, gumguard, nightguard, occlusal splint, bite splint, or bite plane. The American Dental Association (ADA) recommends mouthguards be used in twenty-nine sports: acrobatics, basketball, bicycling, boxing, equestrian, football, gymnastics, handball, ice hockey, inline skating, lacrosse, martial arts, racquetball, rugby football, shot putting, skateboarding, skiing, skydiving, soccer, softball, squash, surfing, volleyball, water polo, weightlifting, and wrestling. Mouthguards may also be used to prevent or reduce the effects of concussions.

### BRIEF SUMMARY

In embodiments, a mouthguard may be worn by a user. The mouthguard includes a body or frame having a channel to receive a user's set of upper or lower teeth, a processor, at least one sensor, a power source, an antenna, an LED display or any combination thereof.

The mouthguard can display a message that can be displayed to others and/or may provide other information—either illustratively via light or via a data stream from an antenna.

In accordance with some embodiments, a mouthguard has a body having a channel to receive a user's set of upper or lower teeth. The mouthguard has a processor, a power source, and a display embedded in the interior of the body.

In another embodiment, a mouthguard has a body having a channel to receive a user's set of upper or lower teeth. The mouth also includes a front face facing away from the teeth in an outward direction so that person can view the front face when installed on the user; a back face opposing the front face; and a interior in between the back face and front face. The mouthguard has a processor, a power source, and a display embedded in the interior of the body. The display is disposed in the front face.

In accordance to another embodiment, a method of making a mouthguard comprising: providing a mold; disposing in the mold: a processor; at least one sensor; a power source; and a display in a front face of the mold, the processor being electronically connected to the display, the at least one sensor, and the power source; after the disposing, filling the mold with a material, thereby encapsulating the display, the processor, the at least one sensor, and the power source in the material.

In accordance to another embodiment, a method of operating a mouthguard includes disposing the mouthguard in a user's mouth, the mouthguard comprising a display, a processor, at least one sensor, and a power source each being encapsulated in the mouthguard; determining, by the at least one sensor disposed in the mouthguard, data; determining, by the processor disposed in the mouthguard, that the data has met a predefined condition; transmitting a signal, by the processor, to the display disposed in the mouthguard to change the display based on a signal associated with the condition being met, the display disposed in the mouthguard in a front face facing away from the teeth in an outward direction so that person can view the display when not obstructed by the user's lips.

### BRIEF DESCRIPTION OF THE DRAWINGS

FIG. 1 depicts a mouthguard and a mouthguard tray according to an exemplary embodiment.

FIG. 2 depicts a main body of a mouthguard according to an embodiment.

FIG. 3 illustrates an exploded view of components of a mouthguard according to an exemplary embodiment.

FIGS. 4A and 4B each depict a mouthguard according to embodiments.

FIGS. 5A, 5B, 5C, and 5D each depict a mouthguard according to exemplary embodiments.

FIGS. 6A and 6B depict a mouthguard with a charging system according to exemplary embodiments.

FIGS. 7A, 7B and 7C depict a mouthguard assembly with a lip guard according to an exemplary embodiment.

FIG. 8 depicts a mouthguard assembly with a lip guard with a helmet attachment according to an exemplary embodiment.

### DETAILED DESCRIPTION OF EMBODIMENTS

The following detailed description includes exemplary implementations of the invention. The description merely defines the general principles of the invention and is not intended to limit the invention, but should include other alternatives of which one of ordinary skill in the art will become aware from an understanding of the details of the following disclosure.

The below description is a general overview of only some embodiments of the mouthguard of the present application. Such mouthguard embodiments of the present application are mouth protectors integrated with technology sources that will not only help prevent dental damage and concussions, but will interact with its host and/or provide a display to other users.

The mouthguard is configured with various functions and capabilities, including some that are unique to mouthguard protection, and will meet or exceed qualifications in today's protection and safety requirements in sports competition.

The mouthguard is available for most contact sports such as basketball, football, soccer, hockey, lacrosse, boxing, and many other sports that require mouthguard protection.

On the field of play or in the home, the mouthguard will give its host accurate measures of: shockload, body temperature, a clear display with LED lighting, read outs of bodily functions (heart rate, etc.), wireless device transmission to a wireless device (e.g., tablet, smart phone, smart watch, laptop, etc.), warning signals (e.g., liquid consumption, over exertion etc.), self-cleaning capabilities, and the like.

Secondary to sports athletes, the mouthguard may also crossover into daily life individuals that require the use of a mouthguard for: teeth grinding, snoring, sleep apnea, and braces.

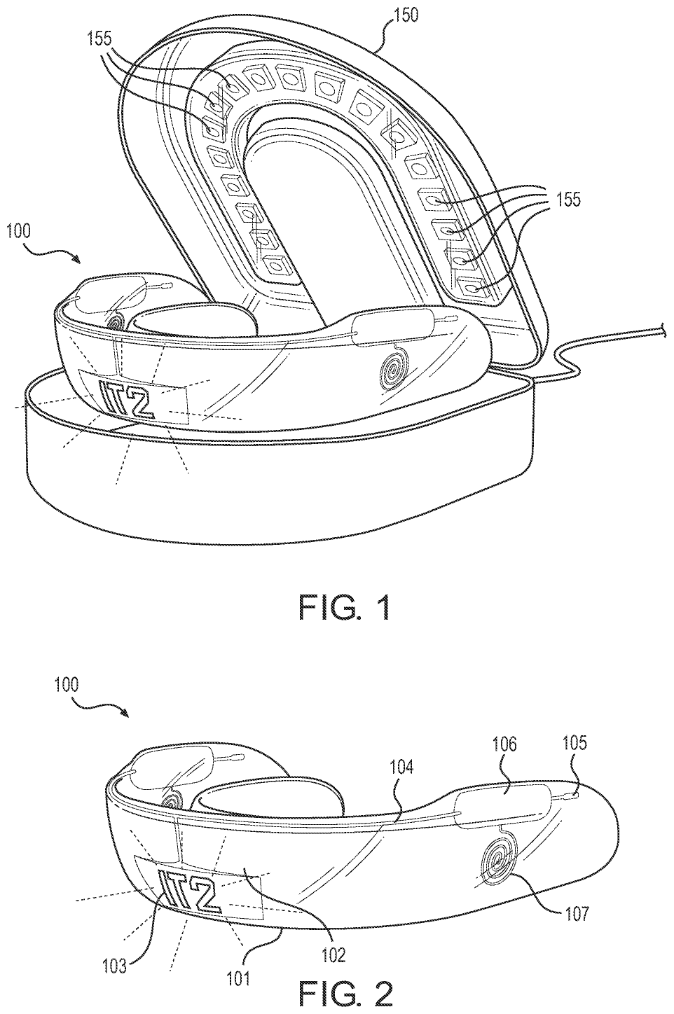

The above are general discussions of the mouthguard embodiments. Referring now to FIGS. 1-8, various embodiments of a mouthguard are shown and described.

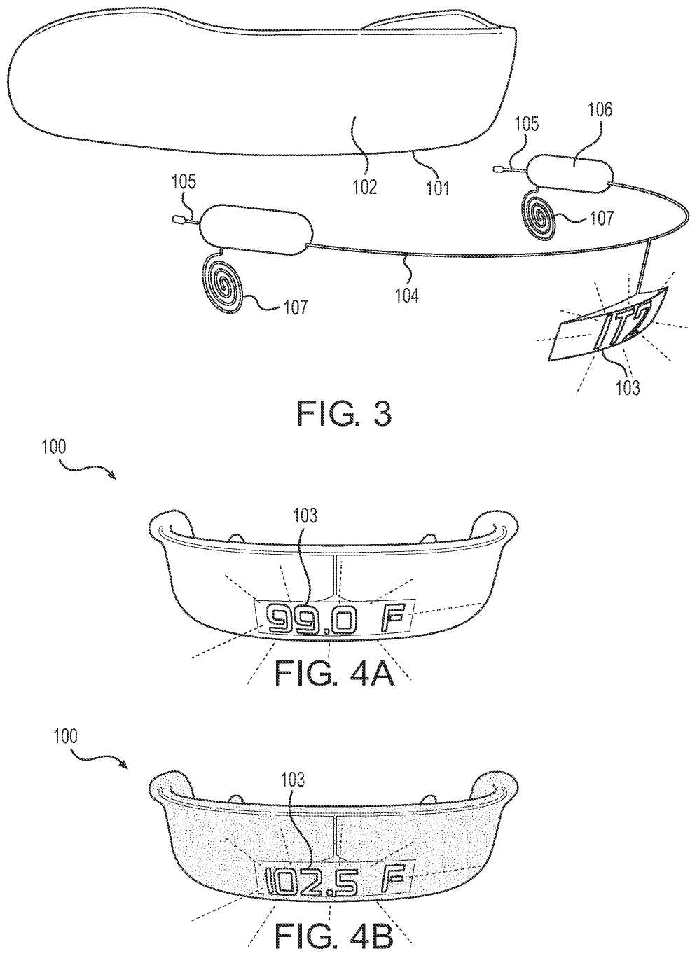

Referring first to FIGS. 1-3, FIG. 1 depicts a mouthguard 100 and a mouthguard tray 150 according to an exemplary embodiment, FIG. 2 depicts a main body 101 of a mouthguard 100 according to an exemplary embodiment, and FIG. 3 illustrates an exploded view of components of a mouthguard 100 according to an exemplary embodiment.

As shown in FIGS. 2 and 3, the mouthguard may include the following: a heat-moldable polymer body 101 which fully encases the following internal components within the body 101: one or more ECUs (Electronic Control Units) 106, batteries (within the ECU 106), charging coils (within the ECU 106), sensors 105, antennas 107, LED matrix display 103.

First, the body 101 (or otherwise referred to herein as “frame”) has a front face 102, a back face opposing the front face, and a channel extended along a centerline of the mouthguard 100. The body or frame 101 can be made of any material to encase one or more of the other components listed above, including ECU, batteries, charging coils, antennas, and LEDs or any combination thereof.

The frame 101 is configured to be inserted into the mouth of a person, either the upper set of teeth or lower set of teeth, according to some embodiments. The frame 101 could be configured to protect the teeth that the frame surrounds in the event that the frame is impacted during an activity, such as an elbow to the mouth or the user hitting their mouth on a hard surface (e.g., floor, head, etc.).

Each of the ECUs 106 may include hardware processors that are configured to process program instructions that may be stored in memory (which may also be stored in the frame). The ECUs 106 may be powered by one or more batteries and receive instructions from memory and/or data from one or more of the sensors. The ECUs 106 each may output data to output devices, such as the LEDs 103, lighting devices within the frame 101, transceivers/antennas 107 to output data, etc. The ECUs 106 also receive data via the antennas 107. The ECUs 106 are configured to execute instructions and operations of the mouthguard 100.

The batteries may be rechargeable power sources for the ECUs 100, power sources for the lighting devices, etc.

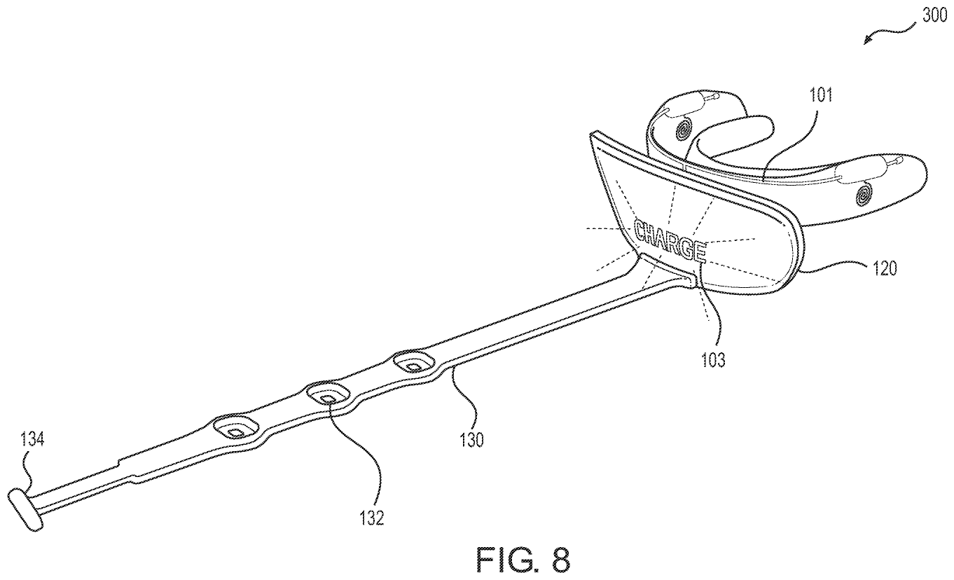

In another embodiment, the mouthguard can connect to a power source outside of the mouthguard, such as a power source located in a different device (e.g., a helmet, wristband, phone, external battery pack, etc.), especially with regard to the embodiment of FIG. 8. The mouthguard can also communicate data between such external devices as well.

The mouthguard could also communicate with speech-to-text communication devices, such as Google voice, Apple's Siri. Amazon's Alexa, etc. so that voice commands to those devices can then control operations in the mouthguard by transmitting commands to the mouthguard. The mouthguard may also have a voice command ECU that receives commands from the user, interprets these commands and then performs control (e.g., instructions discussed herein) within the mouthguard or transmit commands outside of the mouthguard. For example, a user can state a command to the mouthguard while the mouthguard is installed in the users mouth (or outside the users mouth such as on the charger), the ECU process these commands by translating the voice commands to text/instructions, and then the text/instructions is executed to process them to perform a task, similar to Amazon Alexa or Apple Siri. The mouthguard may be connected to Wifi to connect to the internet to perform such operations (in an embodiment where the mouthguard has a transceiver to connect to a router/gateway device to receive an IP address and thus be on a network). In one example, the user can state (while the mouthguard is inside of outside of the user's mouth) “what is the shock load readout” or “what is the hydration levels” and the mouthguard will communicate with the ECU which will receive sensor data (e.g., shock load sensor data, hydration sensor data, etc.) which then may be visually presented in the LED display (or audibly presented using speakers that may be encapsulated in the mouthguard and electronically connected to the ECU).

The sensors 105 may be any type of sensor, such as a temperature sensor, air flow sensor, heart rate sensor, shock sensor or any other sensor. The sensors 105 may be on each side of the ends of the mouthguard, as shown in the embodiment of FIGS. 2 and 3. This may allow the sensors 105 to be placed proximate to the gums of a user or proximate to the user's teeth.

Each sensor 105 is connected to a respective ECU 106 which processes the data. The processing of the data may include determining if a triggering condition has been met. For example, if a user's temperature is greater than 100 degrees (if desired) or increases greater than a preset amount, the mouthguard 100 may display a message on the LED display 103 or light up a colored indicator to light up the mouthguard 100 indicating the user's body temperature is hot. Other conditions may also be displayed such as amount the shock received by the mouthguard is greater than a threshold, the user's heart rate is higher than a threshold, the wetness level is below a threshold, the battery charge is below a threshold, the current status of the mouthguard system meets all operational requirements, etc.

In one embodiment, the mouthguard can use data received to detect sleep apnea (and an air flow sensor can be embedded in the mouthguard as well). The mouthguard can also be used to determine if a user is grinding his teeth at night using the shock sensors.

In one embodiment, there is an LED or series of LEDs 103 within the mouthguard body/frame that shines light within the mouthguard body/frame 101 at the front face 102 so that most of the mouthguard body/frame 101 lights up with the color of the LED/series of LED colors 103. This allows another person looking at the user to view the LED display 103 to see a message through the mouthguard 100.

In another embodiment, the LED/series of LEDs 103 may alternatively or additionally be disposed to be directed at the back face of the LED display/matrix 103 to shine light a direction towards the back face (i.e. in a direction opposite to the direction the LEDs output towards the front face). This would allow a user's mouth to be internally lit up a certain color making the user's mouth look a certain color when the user opens his mouth. In another embodiment, the LED/series of LEDs 103 may be disposed along the wire(s) 104 connecting the ECU 106 and the LED matrix/display 103.

In one embodiment, the mouthguard body/frame 101 may have reflective elements which assist in illuminating the mouthguard when the LED/series 103 of LEDs light up. In any event, light within the mouthguard body/frame 101 reflect within the mouthguard body/frame 101 so that the whole mouthguard body/frame 101 looks like it is illuminating as a whole.

As another example, the sensor 105 may take a heart rate of the user and display the user's heart rate on the LED display 103 and/or transmit this data to a computer/tablet over a short range wireless network (Wifi, Bluetooth, etc.).

As another example, the sensor 105 may take the temperature of the user and display the user's temperature on the LED display 103 and/or transmit this data to a computer/tablet over the short range wireless network (Wifi, Bluetooth, etc.). This is shown in FIGS. 4A-4B.

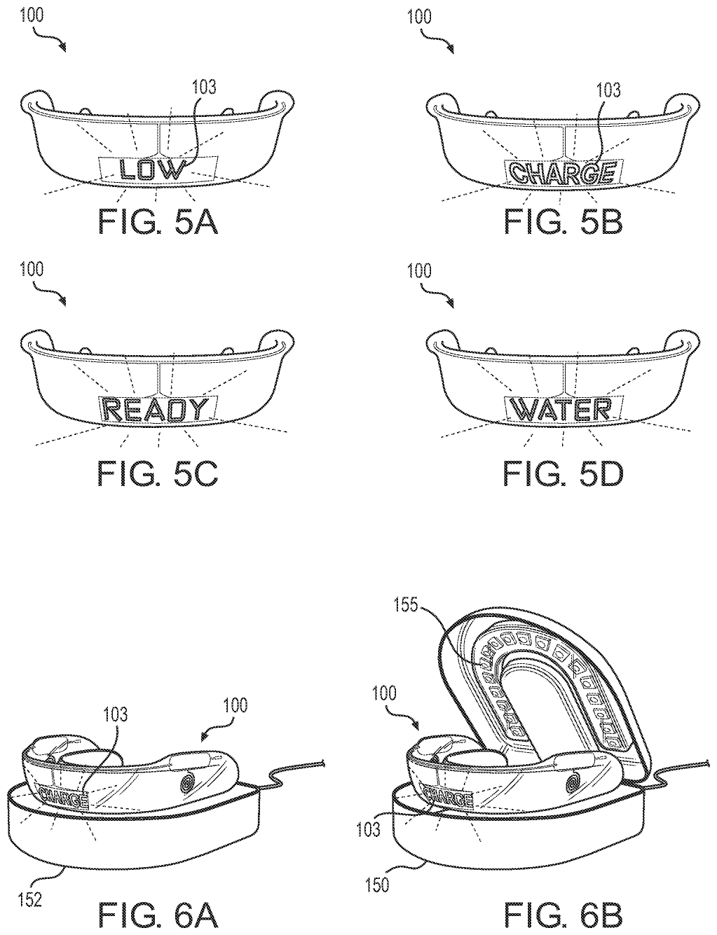

The mouthguard 100 may also display the level of the battery charge, as shown in FIGS. 5A, and as shown in FIGS. 5B, 6 and 7 indicating whether the battery is currently being charged.

Other indications may also be displayed such as “Ready”, which indicates that the mouthguard 100 is sufficiently charged and data can be transmitted, and “Water” which indicates that the mouthguard 100 has detected dehydration symptoms in the user, and it is a warning to the athlete and staff that the subject may be reaching dangerous levels of dehydration and or heat stroke

As mentioned above, the LED display 103 may be positioned in the front part of the mouthguard 100—i.e., where the lips open so that when the lips smile or open, the LED display 103 can be perceived by another user's eyes, etc. The LED matrix 103 may be a series of LED arrays so that any word, number, character or image can be displayed on the LED panel 103. Images can be displayed across the LED matrix 103 via outside devices, but information sent from the mouthguard 100 to the devises cannot be manipulated or changed, in some embodiments, for the safety of the user. This safety mechanism has been implemented so that athletes and staff cannot change data so it appears that the athlete or subject is not in any danger. For home use, doctors or dentist cannot manipulate sleep and teeth grinding results etc.

In some embodiments, operations of the mouthguard 100 (e.g., what is displayed on the LED matrix/display 103, illumination colors, etc.) can be controlled remotely by another user. For example, a team manager can control what is displayed on the LED matrix/display 103 remotely, such as “timeout” or a flame. This allows one to show an active player is doing well (“heat check”) in a game or transmit messages to the players using the mouthguard. This is done using a base computer which connects wirelessly to the mouthguard 100 using short range network protocols (Bluetooth, wifi, etc.) and data can be sent from the base computer and received at the mouthguard 100 and then the ECU 106 of the mouthguard 100 control operations of the mouthguard 100 based on the received instructions.

The antennas 107 may be any transceiver that is configured to connect to another antenna of another computer or electronic device, and the antennas 107 of the mouthguard 100 are configured to transfer data from the mouthguard 100 to the computer or other device. This data can be any data from the sensors 107, status data of a component of the mouthguard 100, or any other data. Included in that data is an alert system with GPS capabilities. The inductive charging base, the chargeable cleaning case, and the mouthguard all have receivers that listen for navigation satellite signals. Once the receiver calculates its distance from multiple satellites, it can then determine where the items are located; this data can then be transmitted to any Wifi or Bluetooth device using an application. This will allow for the user to locate a lost or misplaced mouthguard, charging case, or charging base.

The mouthguard 100 can change from a white or clear translucent to various translucent colors to signal warning conditions, such as high temperature, if a predetermined threshold has been met, as shown in FIGS. 4A and 4B. For example, the sensor 105 in the mouthguard will measure a temperature when the mouthguard 100 is inserted into the mouth, which is an accurate measurement of temperature of the body. A user can set the predefined threshold temperature, at which above, the processor will trigger outputs, such as changing colors of the light in mouthguard 100.

The mouthguard 100 can communicate its hardware status as well as monitored information via its integrated LED matrix display 103, as shown in FIGS. 5A-D.

The mouthguard 100 charges via inductive charging facilitated via a charging platform 152 or while inside its storage case 150. Both of these charging systems 150 and 152 (e.g., charging platform 152 in FIG. 6A and the case 150 in FIG. 6B) can also be used to charge other devices (e.g., tablets, phones, etc.), as they both have built in energy storage units. When the charging systems 150 and 152 are plugged in there is a battery unit that is connected to the main battery or power source so that it can charge the mouthguard even when the charging platform/device is not plugged into a power source. This unit is used for quick charging of devices, and runs on a separate power source as shown in FIGS. 6A and 6B.

The UV LEDs 155 (see FIG. 1) in the case lid act to sterilize the mouthguard and are self-limiting and on a timer. The UV LED's 155 may activate only when the lid is fully closed and will shut off at the end of the timing cycle or when the lid is opened, according to some embodiments. It does this because there are sterilizing UV LEDs 155 in the top and bottom of the charging case as shown in FIGS. 1 and 6B. When the case 150 is closed (i.e., when the mouthguard is in the case 150 and the top of the case is positioned so that the mouthguard cannot exit the case or is completely encompassed within the case), the sterilization light illuminates all of the mouthguard which in turn sterilizes any bacteria or the like on the mouthguard.

The mouthguard 100 may broadcast user information in set intervals (e.g., one second, two second, ½ second, 1 minute, etc.) to help conserve battery life. The ECU 106 is programmable via Bluetooth connection from a smart phone or tablet. The LED matrix 103 display is only enabled when exposed to light—it does not remain on if the users lips are closed, in some embodiments, to conserve power. However, in some embodiments, the LED display 103 is on even when the user's lips and mouth are closed.

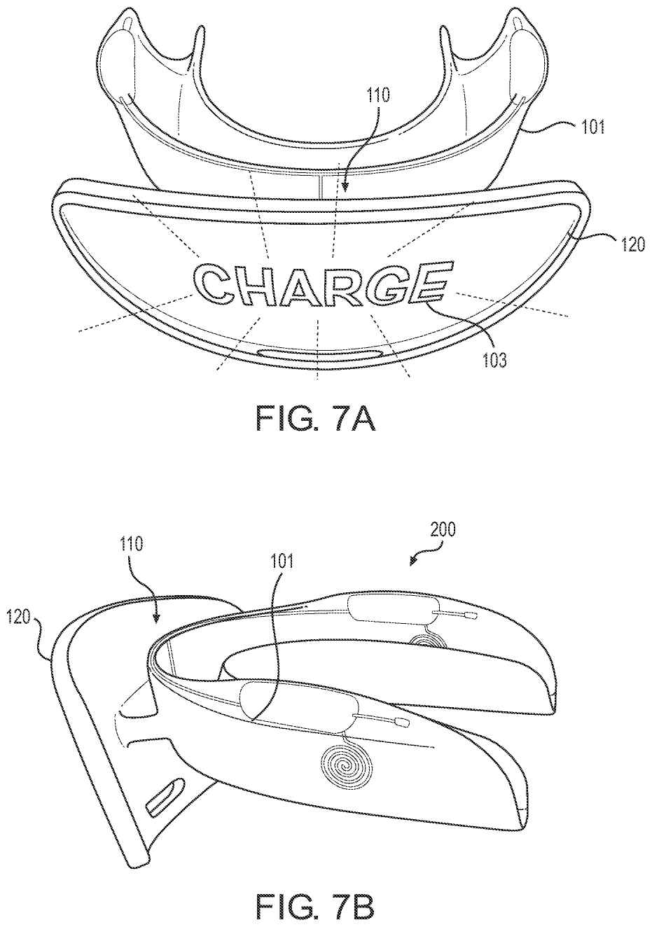

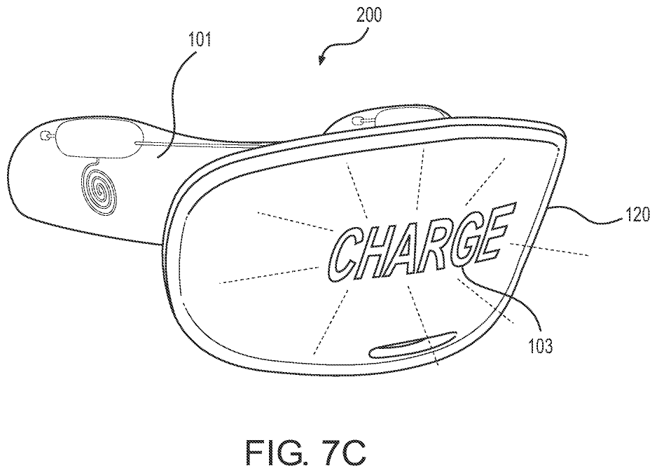

As shown in FIGS. 7A-7C, another embodiment of a mouthguard 200 is shown and may also have the mouthguard 100 discussed above but also a lip guard 120 that is connected to (or integrally formed with) mouthguard 100. The lip guard 120 is spaced an area 110 from the body 101 so that the user's lips can be disposed between the lip guard 120 and the body 101 of mouthguard 100 in order to protect the users lips in the event that the frame is impacted by a hit. The lip guard 120 may be made of similar material to the mouthguard and may be integrally formed with mouthguard 100. As shown in the Figures, the LED display 103 does not have to be displayed in the teeth protection body 101 of the mouthguard but instead can be in the lip protection portion 120 which may be integrally formed with (but spaced from) the body 101 of the mouthguard 100.

As shown in FIG. 8, another embodiment of a mouthguard 300 is shown. The mouthguard 300 may include mouthguard 200 and also may also have a helmet connector 130 that allows the mouthguard 200 to be connected to a helmet (not shown) in the event that the mouthguard is released from the user's mouth. The helmet connector 130 may be made of similar material to the mouthguard and may be integrally formed with the mouthguard. The helmet connector 130 has an end 134 and holes 132. The end 134 is configured to be spaced from holes 132 so that the end 134 wraps around a portion (e.g., a face mask) of the helmet and then is disposed in one of the holes 132 to secure the helmet connector 130 (and thus, the mouthguard 300) to the helmet in the event that the mouthguard 300 is released from the user's mouth.

The above description defines various embodiments of a quick release split drive sprocket assembly, allowing a user to replace a drive sprocket and its corresponding parts easily and efficiently. It should be understood that the foregoing description relates to exemplary embodiments of the invention and that modifications may be made without departing from the spirit and scope of the present invention.

1. A mouthguard comprising:
a body comprising:
a channel to receive a user's set of upper or lower teeth;
a front face facing away from the teeth in an outward direction so that person can view the front face when installed on the user;
a back face opposing the front face;
a interior in between the back face and front face;
a processor;
a power source; and
a display embedded in the interior of the body.

2. The mouthguard of claim 1, wherein the interior is filled with a material, and wherein the display is disposed within the material so that the person can view the display through the front face.

3. The mouthguard of claim 1, wherein the processor and power source are all disposed within the material.

4. The mouthguard of claim 1, further comprising at least one sensor that is a sensor selected from: a temperature sensor, air flow sensor, heart rate sensor, or shock sensor.

5. The mouthguard of claim 1, wherein the at least one sensor is a temperature sensor, and the temperature sensor is communicatively coupled with the processor and power source to deliver temperature information to the processor.

6. The mouthguard of claim 5, wherein the processor is configured to change the display when the temperature measured by the temperature sensor exceeds a threshold.

7. The mouthguard of claim 1, further comprising an antenna that is communicatively coupled to the processor and is configured to communicate data from the mouthguard to a device remote from the mouthguard when the mouthguard is installed in a mouth of the user.

8. The mouthguard of claim 1, wherein the antenna is configured to receive a message from the remote device, wherein the processor is configured to transmit instructions based on the message for the display to display a series of alphanumeric characters.

9. The mouthguard of claim 1, further comprising a charging case which is configured to wirelessly charge the power source.

10. The mouthguard of claim 9, wherein the charging case is configured to completely encapsulate the mouthguard to act as a mouthguard protective case.

11. The mouthguard of claim 1, further comprising a case comprising a series of UV LEDs to sterilize the mouthguard when the case is closed.

12. The mouthguard of claim 1, wherein the series of UV LEDs are organized in a pattern overlapping a centerline along the body of the mouthguard.

13. The mouthguard of claim 1, wherein the display is connected to the power source and processor via a conductor disposed along the body.

14. A method of making a mouthguard comprising:
providing a mold;
disposing in the mold:
a processor;
at least one sensor;
a power source; and
a display in a front face of the mold, the processor being electronically connected to the display, the at least one sensor, and the power source;
after the disposing, filling the mold with a material, thereby encapsulating the display, the processor, the at least one sensor, and the power source in the material.

15. A method of operating a mouthguard comprising:
disposing the mouthguard in a user's mouth, the mouthguard comprising a display, a processor, at least one sensor, and a power source each being encapsulated in the mouthguard;
determining, by the at least one sensor disposed in the mouthguard, data;
determining, by the processor disposed in the mouthguard, that the data has met a predefined condition;
transmitting a signal, by the processor, to the display disposed in the mouthguard to change the display based on a signal associated with the condition being met, the display disposed in the mouthguard in a front face facing away from the teeth in an outward direction so that person can view the display when not obstructed by the user's lips.

16. The method of claim 15, wherein the data comprises temperature data and the at least one sensor is a temperature sensor and the condition is that the temperature has exceeded a predetermined threshold.

17. The method of claim 15, wherein the determining comprising determining, by the at least one sensor disposed in the mouthguard, data;

18. The method of claim 15, wherein the data comprises message data to display on the display and the at least one sensor is a temperature sensor and the condition is that the temperature has exceeded a predetermined threshold.
